(19) United States
(12) Patent Application Publication
(10) Pub. No.: US 2025/0018105 A1
(43) Pub. Date: Jan. 16, 2025

(54) Contrast Injection System and Method for Pre-Stressing Tubing

(71) Applicant: ACIST Medical Systems, Inc., Eden Prairie, MN (US)

(72) Inventors: Yun Zhou, Eden Prairie, MN (US); Vomsheendhur Raju, Fargo, ND (US)

(73) Assignee: ACIST Medical Systems, Inc., Eden Prairie, MN (US)

(21) Appl. No.: 18/220,969

(22) Filed: Jul. 12, 2023

(51) Int. Cl. A61M 5/142

(52) U.S. Cl. CPC A61M 5/142; A61M 2205/50

(57) ABSTRACT

Some contrast injection systems use relatively-large inner diameter tubing to inject contrast fluid into a patient. The contrast injection system of the embodiments presented herein takes advantage of the elastic properties of tubing to “pre-stress” smaller inner diameter tubing into larger inner diameter tubing prior to injecting contrast into the patient. As such, the contrast injection system of these embodiments can reduce the risk of system-pressure-limit-induced injection interruption, improve maximum flow rate, and reduce the use of plastics.

### BACKGROUND

In some imaging modalities, a contrast fluid is used to enhance the contrast of target features in the patient's body. A contrast injection system can inject the contrast fluid into the patient (via tubing) at a certain flow rate and volume. In some injection systems, the contrast fluid is injected using a syringe pump, while in other injection systems, the contrast fluid is injected using a peristaltic pump.

### BRIEF DESCRIPTION OF THE DRAWINGS

FIG. 1 is an illustration of an example syringe-less injection system of an embodiment suitable for a single-use protocol.

FIG. 2 is an illustration of an example syringe-less injection system of an embodiment suitable for a multi-use protocol.

FIG. 3 is an illustration of an example syringe-based injection system of an embodiment.

FIG. 4 is an illustration of an experimental set-up of an embodiment.

FIG. 5 is a graph illustrating peak injector pressure drops using the experimental set-up of FIG. 4.

FIG. 6 is a flow chart of a method of an embodiment for pre-stressing tubing.

FIG. 7 is a diagram illustrating various example ways to restrict a distant end of a patient line.

FIGS. 8A-8C are block diagrams of example injection systems of an embodiment.

FIG. 9 is a flow chart of a method of an embodiment for pre-stressing tubing.

### DETAILED DESCRIPTION

### Introduction

As mentioned above, an injection system can inject the contrast fluid into the patient to enhance the contrast of target features in the patient's body during a scanning procedure. Contrast-enhanced image quality is closely related to contrast delivery output or injection performance, and relatively-large inner diameter plastic tubing may be needed to deliver the contrast fluid at a consistent desired flow rate without exceed a pressure limit of the injection system.

The following embodiments can be used to allow for smaller inner diameter plastic tubing to be used. By requiring less plastic to be used, these embodiments are more cost effective and environmentally sustainable than the system discussed above. In general, these embodiments recognize that the injection system tubing has elastic properties that allow the inner diameter of the tubing to expand when under certain pressure for a certain period of time-and retain that expanded state even when pressure is removed. Taking advantage of this phenomenon, the injection system of these embodiments can be used to expand the inner diameter of the tubing prior to the tubing being used to inject fluid into a patient, thereby transforming tubing with a relatively-small inner diameter to tubing with a relatively-large inner diameter. In one embodiment, the injection system performs this “pre-stressing” process after (or as part of) performing an air purging process on the tubing, as fluid (e.g., saline) is already being applied under pressure to the tubing, and the pressure in the tubing can be advantageously increased to induce stress on the tubing. To achieve the increased pressure in the tubing, for instance, the distal end of the patient line can be restricted to partially or fully block air flow to allow pressure to build-up in the tubing. Various restriction mechanisms are provided.

In one embodiment, an injection system is provided comprising one or more processors, a non-transitory computer-readable medium, and program instructions stored on the non-transitory computer-readable medium. The program instructions, when executed by the one or more processors, cause the one or more processors to: perform an air-purging operation by supplying a fluid at a first pressure into a tubing connected to the injection system; and perform a pre-stressing operation on the tubing by causing pressure in the patient line to increase to a second pressure greater than the first pressure. The pressure in the patient line can be caused to increase to the second pressure in any suitable way. In one example, the pressure can be caused to increase to the second pressure by supplying the fluid at the second pressure (e.g., to expand the inner diameter of the tubing by 5%, a pump can fill an additional 10.25% of tubing's original volume to increase to the second pressure). In another example, the pressure can be caused to increase to the second pressure by restricting a distal end of the tubing so that the fluid pressure increases inside the tubing without necessarily increasing the pressure of the fluid supply.

In another embodiment, a method for pre-stressing a patient line is provided and is performed in an injection system prior to injecting fluid in a patient. The method comprises: applying fluid under pressure to purge air from a patient line; increasing pressure in the patient line to expand an inner diameter of the patient line; and after the inner diameter of the patient line has been expanded, injecting fluid through the patient line and into the patient.

In yet another embodiment, an injection system is provided comprising: means for priming tubing used with the injection system; and means for deforming the tubing by expanding its inner diameter before the tubing is used by the injection system to inject a fluid in a patient.

Other embodiments are possible, and each of the embodiments can be used alone or together in combination.

### Example Injection Systems

The injection of fluids into patients is used in several medical procedures. For example, a contrast agent (or contrast medium) may be injected into a patient to enhance contrast of target (body) features during a scan to make the target features more conspicuous. For example, an iodine-based contrast agent (e.g., comprising iopamidol) is commonly used in computed tomography (CT) applications, such as angiography investigations. The contrast agent is usually injected into a blood vessel of a patient by an injection system. The injection system pressurizes the contrast agent and injects it into the patient's vasculature or organ under predetermined injection conditions (e.g., at a predetermined flow rate and volume).

A powered injection system (sometimes referred to herein as an injector system or injector) can use a syringe or be syringe-less (e.g., using a peristaltic pump with rollers to sequentially and alternately squeeze a tube to control fluid flow). The following paragraphs provide a brief overview of both types of systems. It is important to note that a specific type of system (e.g., syringe or syringe-less) and the details presented below should not be read into the claims unless expressly recited therein.

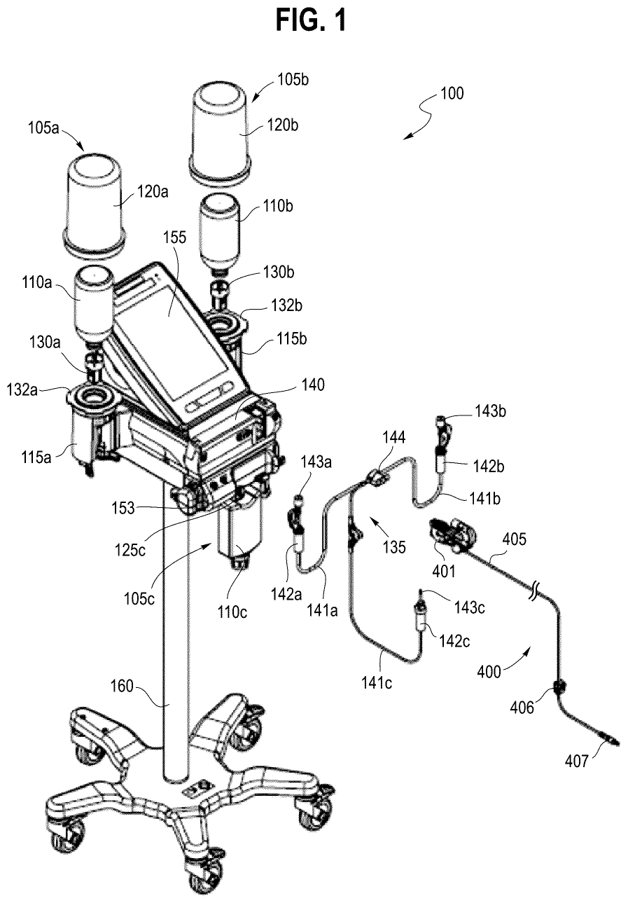

Turning now to the drawings, FIG. 1 is an illustration of a syringe-less injection system 100 of an embodiment. The injection system 100 can be located in a medical suite of a hospital, health care facility, and/or any other facility. The medical suite can comprise a control room where an operator of the injection system 100 may be stationed, as well as a procedure room (such as an imaging room) where the injection system 100 and other equipment related to a medical imaging procedure may be located. In this embodiment, the injection system 100 is supported by a stand 160, which is provided with wheels to facilitate moving the injection system 100; moreover, the wheels can have a foot brake to secure the injection system 100 in position. Alternatively, the injection system 100 can be provided with a boom mount that allows installation of the injection system 100 in the ceiling or wall of the imaging room, or even on the gantry of a CT scanner. The injection system 100 comprises a control unit 155 that controls the operation of the injection system 100 and can comprise a touch-screen and several buttons or other user interface elements, which can be used by an operator to interact with the control unit 155.

As shown in FIG. 1, in this embodiment, the injection system 100 comprises a first supply station 105a, a second supply station 105b, and a third supply station 105c for supplying fluids to be injected from corresponding receptacles. Particularly, the first and second supply stations 105a, 105b supply fluid from bottles 110a, 110b, respectively, and the third supply station 105c supplies a fluid from a pouch 110c. In one example, the first and second supply stations 105a, 105b both hold contrast agent, and the third supply station 105c holds saline solution. In this example, the injector system 100 automatically switches from whichever of the first and second supply stations 105a, 105b is the active/current station to the other station when contrast agent is depleted in the active/current station. In another example, one of the first and second supply stations 105a, 105b holds contrast agent and the other holds saline solution. Other example implementations are possible. Each bottle 110a, 110b may contain a single or multiple doses of different contrast agents or of the same contrast agent. The pouch 110c generally contains a bulk of saline to be supplied before (pre-flush), after (post-flush), or between (interphase) injections of the contrast agent, or alternatively in rapid alternate succession with the contrast agent (to achieve a mixing of the contrast agent and the saline solution within an organ of the patient). In this latter case, the third supply station 105c can be eliminated.

In this embodiment, the first and second supply stations 105a, 105b comprise respective bottle holders 115a, 115b for housing and supporting the bottles 110a, 110b. Protective covers 120a, 120b may be mounted on the bottle holders 115a, 115b to cover the bottles 110a, 110b, thereby defining a (closed) chamber for housing the bottles 110a, 110b. The protective covers 120a, 120b can serve as bottle insulators to help maintain the pre-warmed contrast temperature (in some cases, warm contrast is injected, and contrast bottle is pre-warmed). The supply station 105c comprises a hook 125c for hanging the pouch 110c.

The injection system 100 of this embodiment further comprises a delivery arrangement 135 that determines a fluid pathway for delivering the medical fluids from the receptacles 110a, 110b, 110c to a pressurizing unit 140. The tubing of the delivery arrangement can be made from a plastic material (e.g., PVC). In the first and second supply stations 105a, 105b, bottle connectors 130a, 130b are arranged in connection ports 132a, 132b of the bottle holders 115a, 115b. The bottle connectors 130a, 130b comprise spikes for connecting to the bottles 110a, 110b and connection elements (e.g., a septum or a male Luer lock fitting) in fluid communication with the spikes. The spikes and the connection elements are located at opposite longitudinal ends of the bottle connectors 130a, 130b.

The delivery arrangement 135 (which is sometimes referred to herein as a “day set” or “transfer set”) connects all the supply stations 105a, 105b, 105c to the pressurizing unit 140 for transferring the corresponding medical fluids from the receptacles 110a, 110b, 110c to a patient set assembly 400, which is received inside the pressurizing unit 140. The delivery arrangement 135 comprises a transfer line for each supply station 105a, 105b, 105c. The transfer line of each supply station 105a, 105b comprises a flexible tubing 141a, 141b that is provided (at a distal end thereof with respect to the pressurizing unit 140) with a reservoir (or drip chamber) 142a, 142b and a connection element 143a, 143b for mating with the connection element of the bottle connector 130a, 130b. For example, the connection element 143a, 143b can be a spike connector in case the connection element of the bottle connector 130a, 130b is a septum; alternatively, the connection element 143a, 143b can be a female Luer lock fitting in case the connection element of the bottle connector 130a, 130b is a male Luer lock fitting.

During operation of the injection system 100, the reservoir 142a, 142b and the connection element 143a, 143b are arranged inside the bottle holder 115a, 115b. Analogously, the transfer line of the supply station 105c comprises a flexible tubing 141c that is provided (at a distal end thereof with respect to the pressurizing unit 140) with a reservoir (or drip chamber) 142c and a spike 143c for connecting to the pouch 110c. The flexible tubing 141a, 141b, 141c are coupled (at their proximal ends with respect to the pressurizing unit 140) with a T-connector 144, which comprises a plug for insertion into a corresponding port of the pressurizing unit 140.

The pressurizing unit 140 comprises an electric motor (not shown) that acts on a peristaltic pump that pressurizes the medical fluids (received from the receptacles 110a, 110b, 110c via the delivery arrangement 135) for their injection into the patient (e.g., up to a pressure of 17 bar, or at a flow rate from 0.5 to 9.9 mL/s). The injection system 100 further comprises a patient set assembly 400 which connects the delivery arrangement 135 to the patient for delivering the pressurized fluids thereto. The patient set assembly 400 comprises a delivery tube 405 which is provided (at a proximal end thereof with respect to the pressurizing unit) with a peristaltic pump component 401. The latter is introduced into a dedicated port provided in the pressurizing unit 140, and it is also put in fluid communication with the T-connector 144 of the delivery arrangement 135. The peristaltic pump component 401 houses a rotor having a plurality of squeezing rollers, among which a corresponding portion of a peristaltic tube is inserted, where the peristaltic tube is in fluid communication with the delivery tube 405.

When the patient set assembly 400 is of single use type (as in FIG. 1) for use by a single patient only, the delivery tube 405 can be quite long and provided (at a distal end thereof with respect to the pressurizing unit) with a connection element 407 for mating with a respective corresponding connection element (e.g., a plug) of a vascular access device (VAD) (e.g., IV catheter, peripheral inserted central catheter, etc.) (not shown), which is inserted through the skin into a peripheral vein of the patient to be treated. The delivery tube 405 can also be provided with a clip 406 that pinches the tube and closes the delivery line during installation or uninstallation of the peripheral catheter.

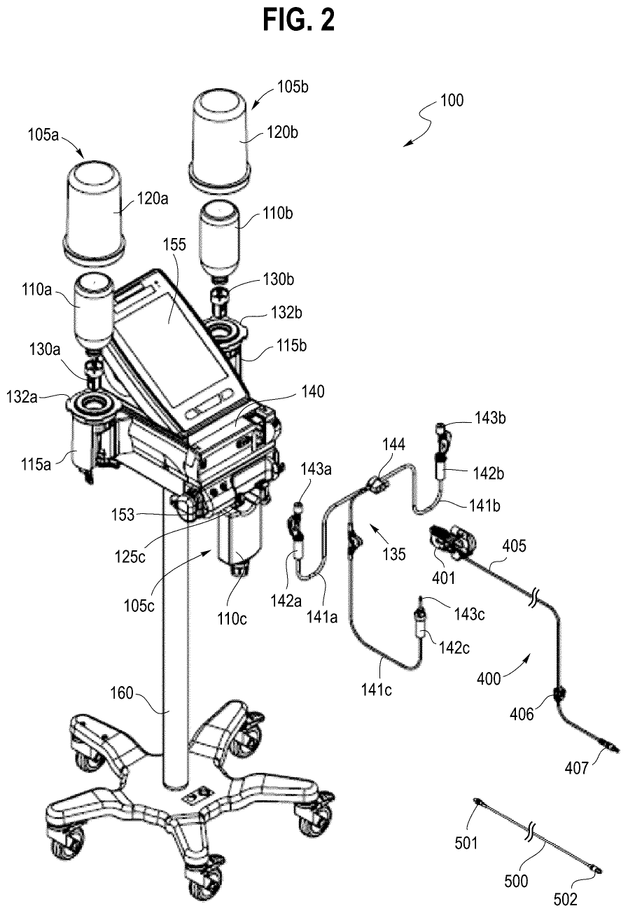

When the patient set assembly 400 is of multiple use type (as in FIG. 2) for use with multiple patients, typically the delivery tube 405 (delivery line) is quite short and is provided at the distal end thereof with a connection element 407 for mating with a corresponding connection element 501 of an additional patient line 500, which typically comprises a quite long flexible tube. The additional patient line 500 terminates (at its distal end) with a connection element 502 for mating with a corresponding connection element possessed by a VAD (not shown).

The patient set assembly 400 is a disposable element, which, in case of single use (FIG. 1), is for use entirely with a single patient, while, in case of multiple use (FIG. 2), it is changed periodically (e.g., every 12 hours), except for the additional patient line 500, which is intended for use with a single patient only and, thus, is discarded at the end of each injection procedure for a given patient, thereby being substituted with a new one when a new patient is ready to be treated.

In both FIGS. 1 and 2, each supply station 105a, 105b, 105c of the injection system 100 further comprises a clamping mechanism (not shown) for engaging the delivery arrangement 135 and, thus, blocking or unblocking the passage of the fluids flowing there through. Specifically, the clamping mechanism of the first and second supply station 105a, 105b can be located inside the bottle holder 115a, 115b, while the clamping mechanism of the third supply station 105c can be located in a dedicated seat 153 housed in the front part of the injector body. Activation (e.g., clamping and de-clamping) of the clamping mechanism can be controlled automatically by the injector software (e.g., as part of the injection steps that are carried out by the injector (according to the injection protocols that are loaded on the injector, typically on the injector remote console not shown in the figures)).

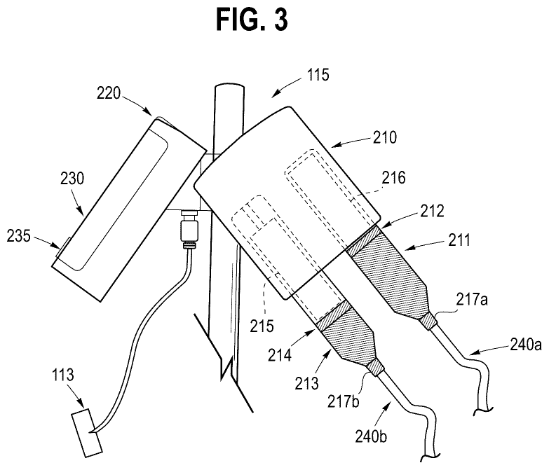

As mentioned above, instead of being syringe-less, an injector system can use a syringe pump to inject fluid to a patient. An example of a syringe-based injector system 115 is illustrated in FIG. 3. As shown in FIG. 3, the injector system 115 of this embodiment comprises an injector portion 210 (capable of operably engaging at least two syringes 211, 213) and a controller device 220 configured to be capable of controlling the dispensing operations of the dispensing medical device. The injector portion 210 also comprises a pair of injector rams 215, 216 adapted to be capable of operably engaging the corresponding pair of syringes 211, 213. The syringes 211, 213 may be attached to the injector portion 210 of the injector system 115 in a fluid-tight manner, and the syringes 211, 213 may be configured to be capable of containing a fluid media (such as a contrast media and/or flushing media).

As shown in FIG. 3, the syringes 211, 213 may further comprise Luer locks 217a, 217b or other fluid-tight connections, so as to allow fluid communication between the syringes 211, 213 and polymeric tubing 240a, 240b configured to be capable of conveying the media contained within the syringes 211, 213 to an injection site (such as an intravenous line) by extending the injector rams 215, 216 into the syringes 211, 213. In addition, the polymeric tubing 240a, 240b may be used to allow fluid communication between the syringes 211, 213 and a container filled with contrast media and/or flushing media such that the syringes 211, 213 may be filled with such media by retracting the plungers 212, 214 into the syringes 211, 213 that may be operably engaged with the injector system 115. Furthermore, the injector rams 215, 216 may be configured to be capable of performing at least one dispensing function (such as extending and/or retracting), so as to be capable of correspondingly advancing and/or retracting the plungers 212, 214 disposed concentrically within the chambers of the syringes 211, 213 to either dispense from or fill the syringes 211, 213 with fluid media. For example, the injector rams 215, 216 may be capable of performing dispensing functions including, but not limited to, extending fully into a syringe 211, 213 so as to initialize the syringe 211, 213 prior to filling the syringe with media; extending into the syringe 211, 213 so as to dispense media from the syringe; and retracting from the syringe 211, 213 so as to fill the syringe with the media contained in a storage container (not shown).

The injector system 115 of this embodiment also comprises a controller device 220 configured to be capable of actuating the injector rams 215, 216 relative to the syringes that may be operably engaged with the dispensing medical device. The controller device 220 may comprise a microprocessor chip or other computer device suitable for controlling the actuation of the injector rams 215, 216, controlling the communication of data between the various components of the injector system 115 and other electronic devices in communication via a wired or wireless network, and/or facilitating the reception of user inputs from a user interface 230. In one embodiment, the controller device 220 comprises one or more processors that are configured to execute computer-readable program instructions stored in a non-transitory storage medium to perform various functions. The user interface 230 can comprise user input elements 235 (such as real or virtual buttons, etc.) to receive user input to, for example, control the dispensing operation of the injection system 115. The injection system 115 can also comprise a hand controller 113 for saline test injection.

It is important to note that the injection systems discussed above are merely examples and that other configurations and types of injection systems can be used. Accordingly, the details presented herein should not be read into the claims unless expressly recited therein.

### Example Set-Up Process

As noted above, one or more sets of plastic tubing can be used with an injection system. For example, one set of plastic tubing (the “day set” or “transfer set”) can connect one or more containers/reservoirs of fluid (e.g., contrast agent and/or saline) to a pressurizing unit in the injector system. Another tubing (a “patient line”) can be used to deliver fluid from the injection system to a patient. More specifically, one end of the patient line can be connected to an output port of the injection system, and the other end of the patient line can be connected to a vascular access device (VAD) in a patient (e.g. a cannula or catheter that is inserted into central or peripheral veins or arteries, that can be implanted or inserted under the skin, allowing fluids and medications to be delivered into a patient's vasculature). The patient line is replaced after each patient use, regardless of single-use or multi-use workflow. The benefit of multi-use workflow for a syringe-based system is that syringes are reused, while in a single-use workflow, syringes are changed after each patient use. While a peristaltic pump system may not have a single-use workflow, the so-called “single-use” and “multi-use” patient sets for a peristaltic embodiment is a design improvement to reuse a peristaltic pump originally designed on a patient set that is single-use. In the following examples, a multi-use workflow is used for the peristaltic pump embodiment. One benefit of a multi-use workflow is rapid patient change (i.e., only changing the patient line instead of the patient line plus syringes and tubing). As another benefit, because less plastic is consumed and replaced, the multi-use workflow is typically less expensive, more environmentally friendly, and more convenient for the health care practitioner (“the user”).

During a set-up process, the user turns on the injection system and sets-up the consumables/disposables. For example, at the beginning of the day, the user can set up the day set (for a multi-use workflow), install the fluid containers (e.g., contrast agent bottle(s) and saline bottle(s)/bag(s)), and interact with the user interface of the injection system to fill the corresponding syringes (in a syringe-based system) that are part of the day set with the fluids. (While a multi-use, syringe-based system is used in this example, as mentioned above, these embodiments can be used with a single-use system and/or with a syringe-less system.) Once the syringes are filled, the user connects one end of the patient line to the distal end of the day set. Before the user connects the other end of the patient line to the VAD, the user primes (or purges) any air present in the tubing. An earlier vacuum process can be performed to remove small air bubbles trapped in syringes and parts of the day set (e.g., manifold connects syringes.

The purging process can be manual or automatic and can be performed in any suitable manner. For example, the user can position the distal end of the patient line (i.e., the end that will eventually be connected to the VAD) over a receptacle to catch any fluid that may flow out of the patient line during the purging process. Then, the user presses the appropriate button on the injector system's user interface to cause fluid (e.g., saline) to flow through the patient line and purge all the air in the line. As another example, the injector system can have an input port to receive the open end of the patient line. In this example, the user merely places the open end of the patient line into the input port of the injection system. Automatically upon detection of the insertion or in response to user input, the injection system can cause fluid to flow into the patient line to start the priming process and later inform the user when air has been purged from the patient line. This eliminates the need for the user to hold the patient line over a receptacle and wait for the purging process to complete (so, the user can attend to other tasks).

At the end of the purging process, fluid completely fills the day set and patient line, and the user connects the patient line to the VAD. After the patient line is connected to the VAD, the user interacts with the user interface of the injection system to start the flow of fluid (e.g., saline or contrast) into the patient.

### Injection Performance Challenges

In general, contrast-enhanced image quality is closely related to contrast delivery output or injection performance, and it is often desired for the injection system to generate a sufficient amount of pressure to deliver a certain volume of contrast to a patient at a certain flow rate (e.g., 8.5 mL/sec) to help ensure accurate imaging. However, injection performance can be limited by many factors, which can be random or unmanageable by an injector system manufacturer. Compromised injection performance can lead to undesirable contrast delivery output, which can decrease image quality or even cause the injection to be aborted, which can disrupt the workflow and might require the user to re-plan the entire procedure, which is undesirable for an imaging laboratory.

Several factors can constrain or limit the flow rate, such as, but not limited to, fluid viscosity, tubing inner diameter and length, pump mechanism and capability, system pressure measurement accuracy, VAD type, length and caliber, and patient condition. For example, achieving an injection rate of 8.5 mL/sec for coronary computed tomography angiography (CCTA) procedures can be challenging with a high-viscosity contrast fluid, such as Iomeron 400 (28.9 cP @ 20° C.) or Visipaque 320 (26.6 cP @ 20° C.) and with patients with small veins requiring relatively-small VADs (e.g., 22 Gauge or 24 Gauge IV catheters or PICC lines). In that situation, the injection system may need to generate higher injection pressure. However, the peak pressure of the injection system can be limited by factors such as pump capability, consumable design, VAD rating, injector control mechanism, and applicable standards/regulations.

Pump type can also affect injector pressure capability. In general, syringe-based pumps can generate higher pressure (e.g., up to 325 pounds per square inch (psi) for a CT injector and up to 1,200 psi for angiography injector), while a peristaltic pump can have difficulty to generate pressure higher than 200 psi. So, where injection protocols demand high injection rates, the injection performance (e.g., the actual flow rate) may be compromised.

Another factor is VAD type, as a VAD can cause a relatively-large pressure drop (e.g., 50 to 100-200 psi) due to its physical characteristics (e.g., a one-inch, 18-22 Gauge cannula). The pressure at the inlet of a VAD is associated with a maximum pressure rating for safety purpose. In addition, the ISO 10555-1 standard requires VAD manufacturers to give user recommendations for the power injector system maximum pressure limit setting, which practically limits the power injector system pressure to be the same as the VAD pressure rating. Since injector system pressure can be defined at the injector side, the pressure at inlet of the VAD must be lower than its rated pressure (i.e., the pressure at the inlet of the VAD equals the pressure set at the system minus the pressure drop through day set and/or patient line).

Yet another factor is fluid pathway resistance through the day set and/or patient line. For example, the inner diameter of the tubing can limit the flow rate, so tubing with a relatively-large inner diameter may be needed to limit the pressure drop from the injection system to the VAD. As another example, the patient line can be relatively long in a multi-use workflow and in a trauma workflow where the patient line is connected to the patient's head. In general, a relatively-long patient line is needed for certain CT scan procedures (e.g., a whole body scan) or if the injector unit cannot be placed close enough to the patient. A longer patient line can result in a greater pressure drop, which can constrain the maximum flow rate of the contrast fluid. Another factor is contrast type and temperature, as viscous, high-concentration contrast can require extra pressure or larger tubing to deliver the contrast at a desired flow rate.

### Using the Elastic Properties of Tubing

Tubing used in an injection system is typically made from polymeric materials, which possesses some elastic properties similar to viscoelastic materials. Because of these stress-relaxation properties, the tubing can deform/expand when stress is applied by injecting fluid in the tubing. With the fluid at a high-enough pressure, the recovery of the strain can be infinitesimally small. That is, the partial recovery of strain can be associated with the elastic deformation, and the unrecovered strain (which is the infinitesimal recovery of the strain happening over a very long period) can be treated as the plastic deformation. (It would take a very long time for plastic tubing to return to its original inner diameter, so the deformation can be considered as permanent for the uses discussed herein.) This effectively increases the inner diameter of the tubing and is analogous to the stress relaxation in polymers. That is, when stress is applied to an extent of constant strain condition as a function of time, the stress diminishes over time to maintain that constant strain state.

The following embodiments take advantage of this phenomenon by “pre-stressing” the tubing (e.g., the patient line and/or day set) before the tubing is used to inject a fluid into a patient. (The terms pre-stressing, pre-stretched, pre-straining, pre-deforming, pre-loading, stress relaxation, and the like may be used interchangeably herein.) That is, a sufficient amount and duration of pressure is applied inside the tubing to cause plastic deformation of the tubing before the tubing is used to inject the fluid into the patient. This pre-stressing effectively increases the inner diameter of the tubing (and may additionally cause decreased friction losses along the tubing), which reduces the pressure drop across it at the time of the first injection to the patient. This is because the laminar fluid flow in a tubing follows Poiseuille's Law:

$$Δ P = \frac{128 * μ * L * Q}{π * {d}^{4}}$$

where “ΔP” is pressure drop,
“μ” is the dynamic viscosity,
“L” is the length of the tube,
“Q” is the flow rate of the fluid inside the tube, and
“d” is the inner diameter of the tube.

As shown by this equation, pressure drop across the tubing is inversely proportional to the fourth power of the inner diameter of the tubing. Thus, a small increment in the inner diameter of the tubing will exponentially decrease the pressure drop for a given flow rate.

This technique of expanding the tubing's inner diameter enhances the injection system's capability and performance without increasing the injection pressure setting limit. Furthermore, the pressure drop can be lower for any given protocol of injection once the tubing is deformed to a larger inner diameter. Other benefits include, but are not limited to: (1) a reduced risk of a system pressure-limit trigger event, (2) a predictable contrast fluid delivery-rate performance, and (3) a cost effective and sustainable (reduced plastic material use) tubing design (i.e., initially-smaller inner diameter tubing can be used, which can result in reduced cost and decreased plastic usage/waste).

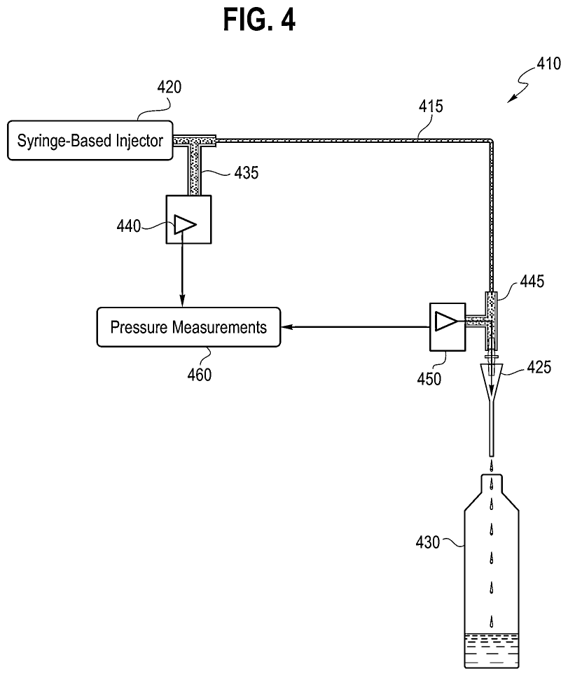

Turning again to the drawings, FIG. 4 is an illustration of an experimental set-up 410 of an embodiment that will be described to illustrate the pressure drop advantage.

As shown in FIG. 4, a patient line 415 connects a syringe-based injector 420 with a 20 Gauge VAD 425 via a male-to-male luer connection. A contrast collection bottle 430 is positioned under the VAD 425 to collect contrast flowing out of the VAD 425 (a patient was not connected to the VAD 425 in this experiment). A first Luer T-connection 435 connects a first pressure sensor 440 to a portion of the patient line 415 near the injector 420, and a second Luer T-connection 445 connects a second pressure sensor 450 to a portion of the patient line 415 near the VAD 425.

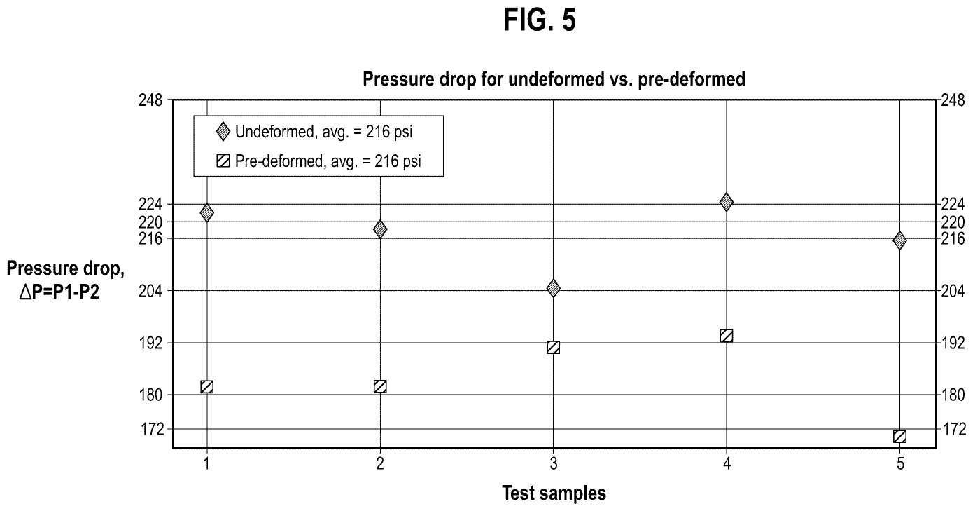

This set-up 410 was used in an experiment conducted to confirm the pressure drop benefit across the patient line 415 with pre-deformation by comparing pressure measurements 460 between the first and second pressure sensors 440, 450 for undeformed and pre-deformed versions of the patient line 415 at a flow rate of 10 mL/sec. The patient line 415 was pre-deformed by performing an injection with a restriction (the VAD 425) at the distal end of the patient line 415 to achieve high injection pressure (close to the maximum injection pressure setting limit) inside the patient line 415 for about 10-12 seconds. Injections were performed by connecting a syringe-based Angio injector (ACIST CVi with an injection pressure setting capability of 1200 psi) to the VAD 425 via the patient line 415. To pre-deform the patient line 415, an injection with a 24 Gauge VAD at 4.5-5 mL/s was performed for 10-12 seconds with a high-viscosity contrast fluid (injector pressure around 450 psi in steady state). Injections with a 20 Gauge VAD at 10 mL/s were performed for ten tubing (five pre-deformed and five un-deformed), and the average differences in the pressure measurements were collected. The graph in FIG. 5 compares the results.

As shown in FIG. 5, the use of a pre-deformed patient line results in a significant pressure drop during injection, which, in turn, improves injection capability. Using a pre-deformed patient line can also have a sustainability benefit. For example, the very large number of contrast procedures performed every year results in a very large amount of plastic waste being generated, and these embodiments can reduce such waste by using a smaller-size patient line. As another advantage, the use of a pre-deformed patient line can result in a smoother workflow with fewer false occlusion alerts which, when they occur, clearly represent an undesired interruption of the injection procedure workflow.

### Example Techniques For Pre-Stressing Tubing

The following embodiments take advantage of the elastic properties of the tubing (e.g., the patient line and day set) by expanding the inner diameter of the tubing before the tubing is used to inject a fluid into a patient (“pre-stressing” the tubing). In one embodiment, the injection system is used to pre-stress the tubing after (or as part of) performing the air purging process, as fluid (e.g., saline) is already being applied under pressure in the patient line during that process, and the pressure can be increased (e.g., up to the pressure rate of the tubing) to induce stress on the tubing. To achieve the increased pressure in the tubing, in one embodiment, the distal end of the patient line is restricted to allow a pressure build-up in the tubing. Various possible restriction mechanisms are discussed below, but any suitable type of restriction mechanism can be used.

As noted above, the tubing can be pre-stressed after or as part of the air purging process. Air is highly compressible, so if there is a large air column in the tubing, it may not be possible to reach pre-stressing pressure even with the injection of the whole syringe volume. So, the pre-stressing would be done after the air purging process in that situation. However, purging and pre-stressing can be done in one action from the user's perspective (e.g., the user may view pre-stressing as part of the air purging process). For example, as will be discussed below, the restriction mechanism can take the form of a small venting hole on a protective cap at the distal end of the patient line. Air can flow through the small venting hole during the purging process. Once air has been purged, a pressure drop through the hole increases as viscosity of fluid is higher than air, and in-line pressure increases, which pre-stresses the tubing. From the user's perspective, the pre-stressing is done in the same process/step as air purging, even though the expansion of the tubing technically occurs after the air has been removed.

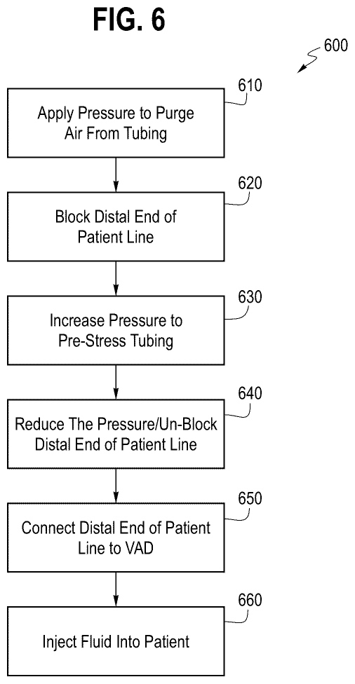

FIG. 6 is a flow chart 600 that illustrates this embodiment. As shown in FIG. 6, the injection system purges air from (“primes”) the tubing (e.g., patient line and day set, if used) by pushing fluid (e.g., saline) through the tubing at a first pressure (act 610). A restriction mechanism is used to partially or fully block the fluid pathway on the patient line (act 620). Then, the injection system pushes the fluid through the patient line at a second pressure, which is higher than the first pressure, for certain time (e.g., seconds to minutes) to pre-stress the tubing (act 630), said second pressure being generated by the restriction mechanism. In one embodiment, the second pressure is lower than but close to the maximum rated pressure of the tubing, although any suitable pressure can be used. The pressure can be estimated by the current/speed of the motor controlling the pump or measured by an external or in-line pressure sensor. After the specified pressure is applied for the specified amount of time, the tubing is pre-stressed. The pressure in the tubing is then reduced (e.g., by the pump releasing the in-line pressure and/or by slowly opening the mechanism that blocked the fluid pathway), and the distal end of the patient line is unblocked (act 640). Then, the distal end of the patient line is connected to the VAD (act 650), and fluid is injected into the patient (act 660). As will be discussed below, one or more of these steps may be performed automatically or semi-automatically by the injection system.

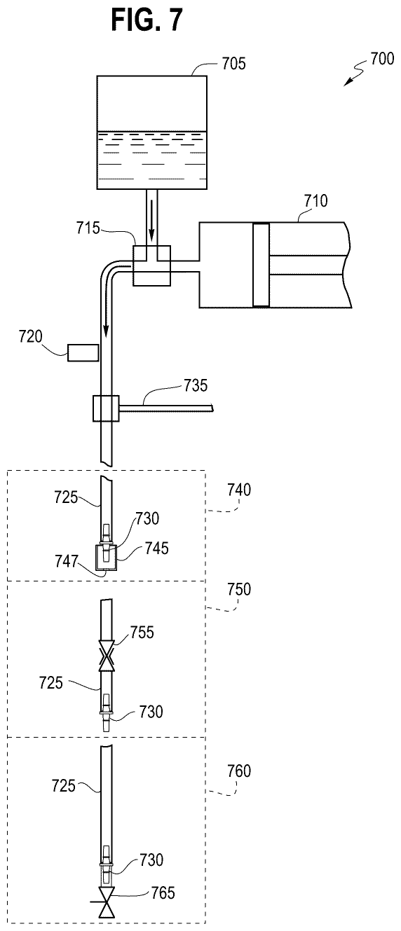

The distal end of the tubing can be restricted in any suitable way, and sections 740, 750, and 760 in FIG. 7 show various example restriction mechanisms that can be used. It should be understood that these are merely examples and that other ways of restricting the distal end of the patient line can be used.

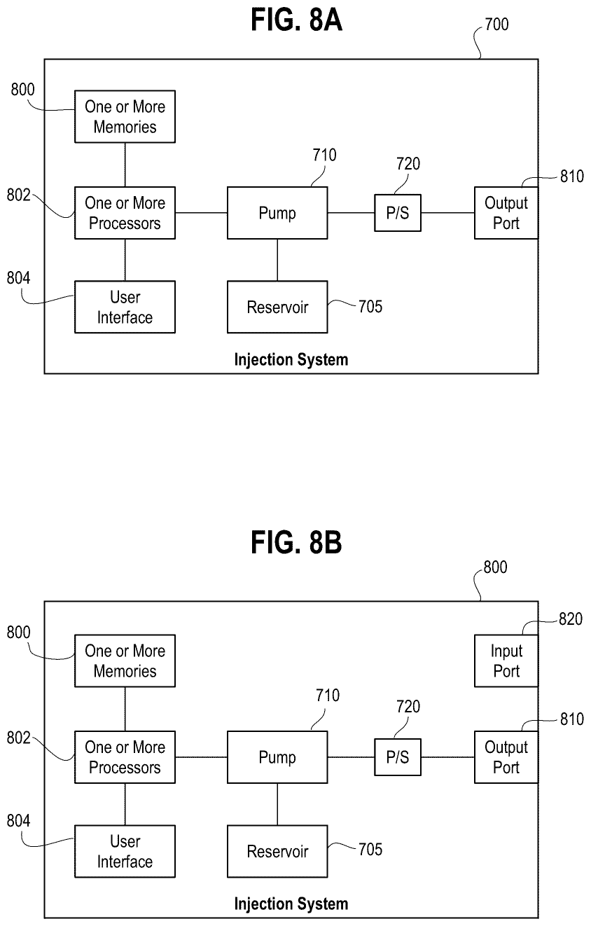

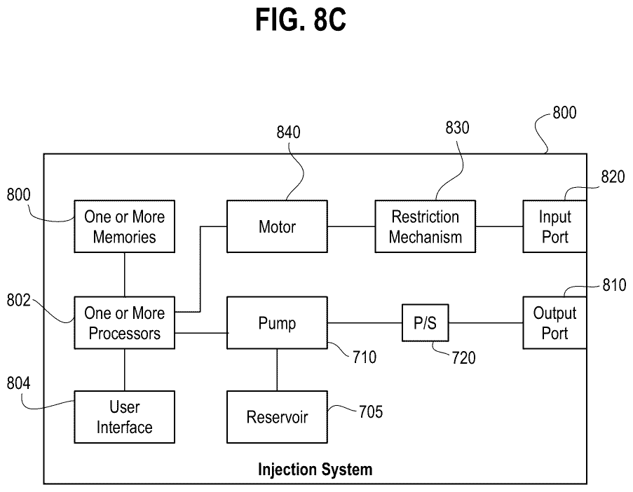

Before turning to these example restriction mechanisms, a brief overview of the injection system 700 used in these examples will be provided. As shown in FIG. 7, in these examples, the injection system 700 comprises a fluid (e.g., saline) reservoir 705 connected with a pump 710 via a valve 715 to control fluid direction. The valve 715 is also connected to day-set tubing (not shown to simplify the drawing). A patient line 725 having a male Luer connector 730 at its distal end connects to an output port 810 (see FIG. 8A) of the injection system 700. Optionally, a valve 735 is used to connect to other fluid reservoirs. Also, an optional pressure sensor 720 can be used. It should also be understood that FIG. 7 is a simple diagram for illustration purposes and that other/different components can be used.

FIG. 8A is a block diagram of the injection system 700 in this example. As shown in FIG. 8A, in addition to the reservoir 705, pump 710, and pressure sensor 720, the injection system 700 of this embodiment comprises one or more volatile or non-volatile memories 800 (e.g., non-transitory computer-readable medium/media), one or more processors 802, a user interface 804 (e.g., a touch screen display, a keyboard, and/or a mouse, etc.), and an output port 810 with an opening sized to accept a proximal end of the patient line 725. In one embodiment, the one or more volatile or non-volatile memories (e.g., RAM, a flash drive, a hard drive, etc.) 800 store program instructions that, when executed by the one or more processors 802, cause the one or more processors 802 to perform the functions described below, as well as potentially other/different functions, if desired. In other embodiments, a pure-hardware implementation (e.g., using logic gates, switches, an application specific integrated circuit (ASIC), etc.) is used. Also, “means” for performing a function can be implemented with one or more processors executing computer-readable program instructions and/or exclusively in hardware.

As mentioned above, sections 740, 750, and 760 in FIG. 7 show various example restriction mechanisms that can be used. Turning first to section 740, in this example, a protective cap 745 with a venting hole 747 is used as the restriction mechanism. Currently, some patient lines are shipped with protective caps on both the distal and proximal ends of the patient line and are used in a sterilization process that occurs at the end of the manufacturing process and prior to the user receiving the patient line. More specifically, patient lines usually have protective caps on both ends to prevent contamination during handling. A small venting hole on the protective cap is used to allow EtO sterilization (providing a fluid path), which is a sterilization method for disposables. The venting hole 747 allows a pathway for the sterilization fluid to pass. In this embodiment, the protective cap 745 is kept on or is placed on the distal end of the patient line 725 (if it was previously removed) for the pre-stressing process and acts as a nozzle that partially blocks the fluid pathway in the patient line 725 to allow pressure to build-up in and pre-stress the patient line 725. Using the protective cap 745 as a restriction mechanism has the advantages of not increasing capital or disposable cost since the protective cap 745 is already supplied with the patient line 725.

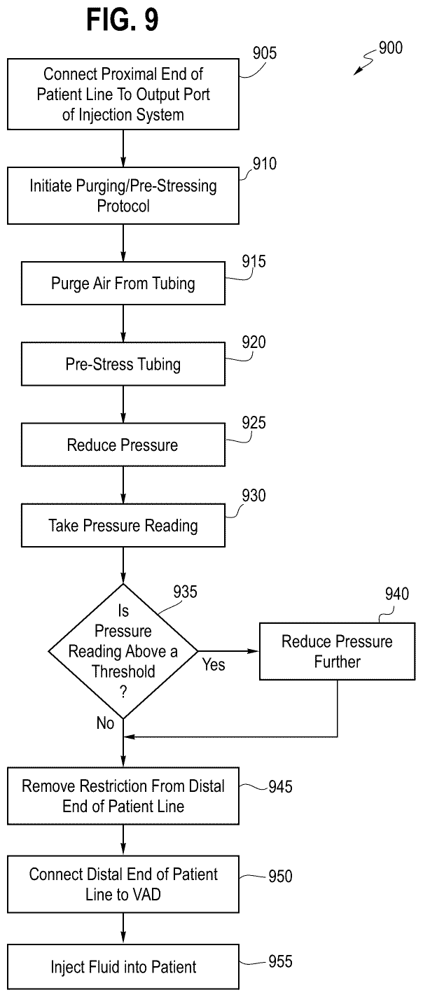

FIG. 9 is a flow chart 900 of an example method of pre-stressing the patient line 725 using the protective cap 745 as the restricting mechanism. As shown in FIG. 9, the user connects the proximal end of the patient line 725 to the output port 810 of the injection system 700 (act 905). In this example, the distal end of the patient line 725 (the end that will eventually be connected to the VAD) is covered with the protective cap 745, and the protective cap 745 is kept on the distal end of the patient line 725 during the air purging process. This example assumes that the venting hole 747 is large enough to allow air to be purged from the patient line 725, albeit with a limited flow rate that may affect performance. Keeping the protective cap 745 on the distal end of the patient line 725 during the air purging process results in a more-efficient workflow.

Next, the user initiates the purging/pre-stressing protocol by interacting with the user interface 804 (act 910). Alternatively, the purging/pre-stressing protocol can be automatically triggered by an event, such as the installation of the patient line. In response, the processor(s) 802 cause the pump 710 to pump fluid (e.g., saline) from the reservoir 705 into the patient line 725 at a sufficient pressure to purge air from the patient line 725 (act 915). In one example, the pressure is lower than about 50 psi. In another example, the pressure is sufficient to result in a flow rate of about 4-6 mL/s. During this process, the user can hold the distal end of the patient line 725 over a receptacle to catch any fluid that may flow out of the patient line 725 during the purging process.

After the air is purged from the patient line 725, the injection system 700 can automatically (or in response to an input from the user via the user interface 804) transition to the pre-stressing process and control the pump 710 to increase the pressure for a certain time to pre-stress the patient line 725 (act 920). In one embodiment, the pressure applied to pre-stress the patient line 725 is higher than the pressure applied to purge air from the patient line 725 but not greater than an upper limit of the tubing's pressure rating determined by the manufacturer. In one example, the pressure is greater than about 50 psi. In another example, the pressure is greater than about 100 psi (e.g., about 300 psi) and applied for about 30 seconds. In yet another example, the pressure is about 400 psi (e.g., when a VAD pressure rating is about 325 psi and pressure measurement uncertainty is 50-75 psi). It should be noted that these are merely examples, and any suitable pressure can be used and may be based on the type of restriction mechanism and patient line design. It should also be noted that while these examples were described in terms of the patient line, the pre-stretching effect would also be applied to the day set tubing.

After the increased pressure has been applied to the patient line 725 for the prescribed period of time, the processor(s) 802 can cause the pump 710 to reduce pressure in the patient line 725 (act 925). The processor(s) 802 can also take a pressure reading of the pressure sensor 720 to determine if there is any residual pressure in the patient line 725 that can cause significant leakage of fluid from the distal end of the patient line 725 when the protective cap 745 is removed (acts 930 and 935). If the residual pressure is above a threshold, the processor(s) 802 can automatically control to the pump 710 to reduce the pressure further (act 940), e.g. by acting on the pump. For example, residual pressure can be released by slightly pulling back on the syringe. If the restrictive mechanism is the venting hole on the protective cap, no action may be needed as the residual pressure will gradually drop. If the restrictive mechanism is a pinch valve, for example, residual pressure may be released by pump side. After pressure is released (or if the residual pressure is not above the threshold), the user removes the protective cap 745 from the distal end of the patient line 725 (act 945) and connect the distal end of the patient line 725 to the VAD (act 950). The injection system 700 would then start injecting fluid into the patient (e.g., in response to a command from the user) (act 955).

During the purging and pre-stressing processes, the user can hold the distal end of the patient line 725 over a receptacle to catch any fluid that may flow out of the patient line 725. In another embodiment (shown in FIG. 8B), the injection system 800 has an input port 820 with an opening sized to accept the protective cap 745. Here, the female opening of the input port 820 is larger than the female opening of the output port 810, since a protective cap would not be on the proximal end of the patient line 725 going into the output port 810. In another implementation, two input ports can be provided-one sized for the protective end cap 725 and the other sized for the distal end of the patient line 725-to account for either possibility. In yet another implementation, the input port 820 can have an adjustable female opening to account for either possibility. Other accommodations are possible. With any of these implementations, the user can place the distal end of the patient line 725 in the input port, so he can attend to other tasks during the air purging/pre-stressing processes. The injection system 800 can have a receptacle positioned to catch any fluid that may flow out of the patient line 725 during the air purging/pre-stressing processes.

As noted above, restriction mechanisms other than the protective end cap 745 can be used. For example, as shown in section 750 of FIG. 7, a pinch valve 755 can be used. Although the pinch valve 755 is not shown as positioned at the very end of the patient line 725 in the diagrammatic illustration in FIG. 7, it is still considered to be “at the distal end of the patient line 725”, as that phrase is used herein. However, it may be desired to place the pinch valve 755 as close to the very end of the patient line 725 as possible to help ensure there is not a significant decrease in the inner diameter of the tubing between the pinch valve 755 and the very end of the patient line 725. As yet another example, (shown in section 760), a (disposable) valve (e.g., a stopcock) 765 connected to the Luer connector 730 of the patient line 725 is used. In this alternative, the valve 765 can be connected to the patient line 725 prior to the air purging operation (with the valve 765 being open during the air purging operation and then closed for the pre-stressing operation), or the valve 765 can be connected to the patient line 725 after the air purging operation.

In one embodiment, the user manually closes the pinch valve 755 or stopcock 765 after the air purging process and prior to the start of the pre-stressing operation. In another embodiment (see FIG. 8C), the restriction mechanism 830 (e.g., pinch valve, stopcock, etc.) is part of the injection system 800 and is actuated by a motor 840 controlled by the processor(s) 802 in the injection system 800. In some embodiments, the air purging and pre-stressing are performed automatically once the user inserts the distal end of the patient line 725 into the input port 820 of the injector system 800. In other embodiments, the user would interact with the user interface 804 of the injector system 800 to initiate the processes or to enable/disable the automatic functionality. Either way, eliminating the need for the user to hold and interact with the patient line 725 and the restriction mechanism during the air purging and pre-stressing processes provides an improved workflow for the user, as the user is able to attend to other tasks while these processes are being performed.

### CONCLUSION

Various examples of systems, devices, and/or methods are described herein. Any embodiment, implementation, and/or feature described herein as being an “example” is not necessarily to be construed as preferred or advantageous over any other embodiment, implementation, and/or feature unless stated as such. Thus, other embodiments, implementations, and/or features may be utilized, and other changes may be made without departing from the scope of the subject matter presented herein.

Accordingly, the examples described herein are not meant to be limiting. It will be readily understood that the aspects of the present disclosure, as generally described herein, and illustrated in the figures, can be arranged, substituted, combined, separated, and designed in a wide variety of different configurations.

Further, unless the context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Further, terms such as “A coupled to B” or “A is mechanically coupled to B” do not require members A and B to be directly coupled to one another. It is understood that various intermediate members may be utilized to “couple” members A and B together.

Moreover, terms such as “substantially” or “about” that may be used herein, are meant that the recited characteristic, parameter, or value need not be achieved exactly but that deviations or variations, including, for example, tolerances, measurement error, measurement accuracy limitations and other factors known to a skill in the art, may occur in amounts that do not preclude the effect the characteristic was intended to provide.

It is intended that the foregoing detailed description be understood as an illustration of selected forms that the invention can take and not as a definition of the invention. It is only the following claims, including all equivalents, that are intended to define the scope of the claimed invention. Finally, it should be noted that any aspect of any of the embodiments described herein can be used alone or in combination with one another.

What is claimed is:

1. An injection system comprising:
one or more processors;
a non-transitory computer-readable medium; and
program instructions stored on the non-transitory computer-readable medium that, when executed by the one or more processors, cause the one or more processors to:
perform an air-purging operation by supplying a fluid at a first pressure into a tubing connected to the injection system; and
perform a pre-stressing operation on the tubing by causing pressure in the patient line to increase to a second pressure greater than the first pressure.

2. The injection system of claim 1, wherein the tubing comprises a patient line and/or a day set.

3. The injection system of claim 1, wherein the tubing comprises a patient line, and the injector system further comprises a port configured to receive a distal end of the patient line.

4. The injection system of claim 3, wherein the port is sized to receive a protective cap on the distal end of the patient line.

5. The injection system of claim 3, further comprising:
a motor; and
a restrictor actuatable by the motor;
wherein the program instructions, when executed by the one or more processors, further cause the one or more processors to:
control the motor to actuate the restrictor to restrict flow out of the distal end of the patient line during the pre-stressing operation.

6. The injection system of claim 5, wherein the restrictor comprises a pinch valve.

7. The injection system of claim 5, wherein the restrictor comprises a stopcock.

8. The injection system of claim 1, wherein the injector system is syringe based.

9. The injection system of claim 1, wherein the injector system is syringe-less.

10. The injection system of claim 1, wherein the program instructions, when executed by the one or more processors, further cause the one or more processors to:
perform the air-purging operation and/or the pre-stressing operation automatically, thereby allowing a user to attend to another task during the air-purging operation and/or the pre-stressing operation.

11. A method for pre-stressing a patient line, the method comprising:
performing in an injection system prior to injecting fluid in a patient:
applying fluid under pressure to purge air from a patient line;
increasing pressure in the patient line to expand an inner diameter of the patient line; and
after the inner diameter of the patient line has been expanded, injecting fluid through the patient line and into the patient.

12. The method of claim 11, further comprising:
restricting flow out of a distal end of the patient line to increase the pressure in the patient line.

13. The method of claim 12, wherein restricting the flow comprises actuating a valve to at least partially close off the distal end of the patient line.

14. The method of claim 13, wherein the valve comprises a pinch valve.

15. The method of claim 13, wherein the valve comprises a stopcock.

16. The method of claim 12, wherein a cap on the distal end of the patient line restricts flow out of the distal end of the patient line.

17. The method of claim 11, wherein the pressure in the patient line is increased to at least about 300 psi.

18. The method of claim 11, wherein the pressure in the patient line is increased to at least about 400 psi.

19. An injection system comprising:
means for priming a tubing used with the injection system; and
means for deforming the tubing by expanding its inner diameter before the tubing is used by the injection system to inject a fluid in a patient.

20. The injection system of claim 19, further comprising:
means for deforming comprises means for restricting a flow out of the tubing.
